(19) United States
(12) Patent Application Publication
(10) Pub. No.: US 2010/0285607 A1
(43) Pub. Date: Nov. 11, 2010

(54) METHOD FOR DETECTING AND QUANTIFYING ANALYTES ON A SOLID SUPPORT WITH LIPOSOME-ENCAPSULATED FLUORESCENT MOLECULES

(75) Inventor: Herve Volland, Orsay (FR)

Correspondence Address: OBLON, SPIVAK, MCCLELLAND MAIER & NEUSTADT, L.L.P., 1940 DUKE STREET, ALEXANDRIA, VA, 22314, US

(73) Assignee: COMMISSARIAT A L'ENERGIE ATOMIQUE, Paris (FR)

(21) Appl. No.: 12/668,205

(22) PCT Filed: Jul. 8, 2008
(86) PCT No.: PCT/IB08/02731

§ 371 Date: May 18, 2010

(30) Foreign Application Priority Data
Jul. 9, 2007 (FR) 0704954

(51) Int. Cl. G01N 33/53; G01N 21/76

(52) U.S. Cl. 436/501; 436/172

(57) ABSTRACT

The present invention relates to an improved method for detecting and quantifying analytes on a solid support, with liposome-encapsulated fluorescent molecules.

The present invention relates to an improved method for detecting and quantifying analytes on a solid support, with liposome-encapsulated fluorescent molecules.

Liposomes are particular structures consisting of a vesicle formed by a lipid bilayer and molecules such as for example antibodies, capable of complexing an analyte A, which are coupled to the outer face of the vesicle by covalent bonding. In the case of immunoassays, these structures are used as colorimetric or fluorescent labels after encapsulation, in the vesicle, of a coloured molecule which is most commonly fluorescent, in particular sulphorhodamine. This type of structure makes it possible to have a label with a high specific activity in so far as several thousand or even million coloured molecules are included per liposome. These structures are used in several assay formats: microtitration plate, capillary and immunochromatography (“strip”).

Microtitration-plate or capillary detection of analytes most commonly uses the fluorescent property of the molecule. However, the concentration of the molecule in the liposomes is such that there is a phenomenon of self-quenching of the fluorescence. In order to reveal the fluorescence, it is therefore necessary to dilute the molecule in the medium by destroying the liposomes with a detergent (SINGH et al., Anal. Chem., 72: 6019-6024, 2000; DECORY et al., Appl Environ. Microbiol., 71(4): 1856-1864, 2005; HO et al., Anal. Biochem., 330: 342-349, 2004).

Detection of analytes by immunochromatography (or the strip format) is a technique used in medical diagnosis and new fields of application, such as, for example, the detection of pollutants in the environment. In this format, only the density of the coloration of the molecule encapsulated in the liposomes is used (U.S. Pat. No. 4,703,017; HO and HSU, Anal. Chem., 75: 4330-4334, 2003; PARK and DURST, Anal. Biochem., 280: 151-158, 2000; AHN-YOON et al., Anal. Bioanal. Chem., 378: 68-75, 2004; AHN-YOON et al., Anal. Chem., 75: 2256-2261, 2003). In fact, after migration of the sample on the strip, the liposomes cannot be broken up using detergents, since such an action would result in the signal being diluted over the entire strip by capillary action. Thus, when molecules are sometimes used for their fluorescent properties in the strip format, they are fluorophores which are either directly bound to the antibody by covalent coupling (KIM et al., Environ. Sci. Technol., 37: 1899-1904, 2003) or bound to the antibody via a polymer such as polysaccharides (U.S. Pat. No. 5,543,332).

The advantages of the strip format, result obtained rapidly and easy to use, mean that it is perfectly suitable for “home test” applications or applications in the field. However, its low sensitivity compared with other techniques, such as immunoassays or mass spectrometry, limits its use.

Thus, for some years, new labels for obtaining a colorimetric or fluorescent signal have been developed in order to improve the sensitivity of detection. However, to date, no method exists for detecting analytes on a solid support using the fluorescent properties of molecules encapsulated in liposomes and which makes it possible to obtain a detection sensitivity at least equivalent to that of the other methods of detection carried out on the known solid supports of the prior art (colorimetry, luminescence possibly revealed by a photographic film, etc.).

Thus, the inventors gave themselves the objective of providing a new improved method for detecting analytes on a solid support which makes it possible to exploit the fluorescence properties of molecules encapsulated in liposomes.

This method is based on the use of liposomes containing fluorophores as fluorescent tracers in assays on a solid phase, such as a membrane or a microtitration plate, after drying of these ones. In general, the high concentration of the fluorophores in the liposomes induces a phenomenon of self-quenching of the fluorescence and therefore a weak fluorescent signal. On the other hand, gradual drying of the solid phases will cause lysis of the liposomes without the addition of a liquid detergent, hence a controlled diffusion of their fluorophore content on the solid support, and therefore a decrease or even disappearance of the self-quenching phenomenon, which will be reflected by a large increase in fluorescence.

A subject of the present invention is therefore a method for detecting at least one analyte A in a biological sample, on a solid support, with liposomes encapsulating fluorescent molecules, which method is characterized in that it comprises the following steps:

1) either (a) i) bringing said biological sample capable of comprising at least one analyte A into contact, in the liquid medium, with at least one liposome encapsulating a fluorescent molecule and the surface of which is coupled to molecules capable of complexing said analyte A, then ii) bringing said medium into contact on a solid support containing at least one immobilized substrate capable of binding the analyte A/liposome complexes possibly formed,

or b) bringing said biological sample pre-immobilized on a solid support, said sample being capable of comprising at least one analyte A, into contact with a liquid medium comprising said liposome encapsulating a fluorescent molecule and the surface of which is coupled to molecules capable of complexing said analyte A;

2) removing the liquid medium present outside the liposomes by drying the said solid support;

3) lysing the liposomes; and

4) detecting the fluorescent signals possibly emitted.

According to step 2) of the method in accordance with the invention, the drying of the support will cause the liquid medium outside the liposomes to be removed, and then, in step 3), will cause the liposomes to be lysed without the need to add a liquid detergent, and their fluorophore content to be released while at the same time preventing their diffusion over the entire solid support.

This method thus makes it possible to increase the signal obtained and therefore the sensitivity of the immunoassays (approach 1)(a)), in particular immunochromatographic immunoassays, but at the same time remains simple and quick to use. In the case of immunoblotting (approach 1)(b)), this method makes it possible to obtain a better band definition and a sensitivity that is at least equivalent to that obtained with peroxidase, which is the reference marker. This method also makes it possible to simultaneously analyse several analytes using various populations of liposomes, each population consisting of liposomes, the surface of which is coupled to molecules capable of complexing a particular analyte A and which fluoresce at a particular wavelength depending on the nature of the fluorescent molecule that they encapsulate. In addition, in these two applications, the step of drying the solid support will make it possible to easily preserve the results that it contains.

According to another particular embodiment of the present invention, said step 1)(a)(ii) is carried out:
either by migration of said medium on said solid support due to capillary action;
or by deposition of said medium onto said solid support.

According to yet another particular embodiment of the present invention, said step 1)(b) comprises the following substeps:

i) separating at least one analyte A from said biological sample by electrophoresis, and immobilizing said analyte A on a solid support by electrotransfer;

ii) bringing said solid support on which said analyte A is immobilized into contact with a liquid medium comprising at least one liposome encapsulating a fluorescent molecule and the surface of which is coupled to molecules capable of complexing said analyte A.

According to a particular embodiment of the present invention, said detection method may also comprise, between steps 1) and 2), at least one step of washing the solid support in order to remove the noncomplexed liposomes.

According to the invention, the solid support is preferably chosen from nitrocellulose membranes, microtitration plates, preferably made of polystyrene, and biochips.

According to the invention, the molecules coupled to the liposomes and capable of complexing an analyte A are preferably chosen from primary and secondary antibodies capable of complexing said analyte, a receptor for said analyte, polynucleic acids (DNA or RNA), peptide nucleic acids, lectin, and a carrier protein or chemical molecule (chelate, synthetic receptor) for said analyte. These liposomes may fluoresce at various wavelengths depending on the nature of the fluorescent molecules that they encapsulate.

According to the invention, the liposome-encapsulated fluorescent molecules are preferably chosen from the following fluorophores: phycoerythrin, phycocyanin, fluorescein and its derivatives such as fluorescein isothiocyanate (FITC), rhodamine and its derivatives such as sulphorhodamine and tetramethyl rhodamine isothiocyanate (TRITC), water-soluble derivatives of rhodamine which are in the form of an N-hydroxysuccinimide ester, such as the products sold under the trade name Alexa Fluor® by the company Invitrogen, for example Alexa Fluor® 488, 500, 514, 532, 546, 555, 568, 594, 610-X, 633, 647, 660, 680, 700, 750 and 790, coumarins and their derivatives such as 7-aminocoumarin, fluorescent cyanins such as those sold under the references Cy3, Cy3.5, Cy3B, Cy5, Cy5.5 and Cy7 by the company GE Healthcare, sulphorhodamine derivatives such as sulphorhodamine 101 sulphonyl chloride, also known as Texas Red®, the dyes sold under the trade name Fluoroprobes®, and green fluorescent protein (often abbreviated to GFP), etc. According to the invention, sulphorhodamine and fluorescent cyanins such as Cy5® are particularly preferred.

According to the invention, step 2) of removing the liquid from the solid support is carried out by drying the solid support at a temperature preferably ranging from 20 to 90° C. approximately, and even more preferably at a temperature of approximately 40° C.

According to a preferred embodiment of the invention, the drying step 2) is carried out in an oven at a temperature of from 20 to 90° C. approximately, preferably at a temperature of approximately 40° C.

According to the invention, the duration of the drying step 2) can vary according to the amount of liquid medium present on the support outside the liposomes. This period must correspond to the time necessary for complete drying of the support, but which does not cause lysis of the liposomes. By way of indication, this period can range from 1 to 60 minutes approximately; preferably, the drying period is approximately 5 minutes.

According to a particularly preferred embodiment of the invention, the drying of the support in step 2) is carried out in an oven, at a temperature of approximately 40° C., for a period of approximately 5 minutes.

The lysis of the liposomes in step 3) can be carried out by any suitable technical means, provided that this means does not cause any re-moisturization of the support (thermal, physical or chemical means).

According to a preferred embodiment of the invention, the liposome lysis step 3) is carried out by additional drying of the support. In this case, this additional drying step is generally more rapid than the first drying step 2) required to remove the liquid medium present outside the liposomes. This period can range from 1 to 15 minutes approximately; preferably, the additional drying period in step 3) is approximately 1 minute.

According to a preferred embodiment of the invention, additional drying step 3) is carried out in an oven at a temperature of from 20 to 90° C. approximately, preferably at a temperature of approximately 40° C.

According to a particularly preferred embodiment of the invention, the drying of the support in step 3) is carried out in an oven, at a temperature of approximately 40° C., for a period of approximately 1 minute.

According to the invention, the substrate(s) immobilized on the solid support is (are) preferably chosen from said analyte A to be assayed or an analogue or a fragment thereof, anti-analyte A antibodies, and antibodies against immunoglobulins, for example from mouse, human, rat, goat, etc.

The detection of the analyte A is carried out by reading the fluorescent signals possibly emitted by the liposomes on the solid support. The longer the period for reading the fluorescence emitted, the more the fluorescent signals increase, thereby making it possible to increase the limit of detection. Thus, it is possible to modulate the reading time according to the results obtained and the desired detection limits. According to a preferred embodiment of the method in accordance with the invention, the detection of the fluorescent signals of step 4) is carried out for a period of at least 1 minute, even more preferably for a period ranging from 1 to 5 minutes. For example, when sulphorhodamine is used as fluorophore, the reading time at 600 nm can range from 1 to 5 minutes approximately.

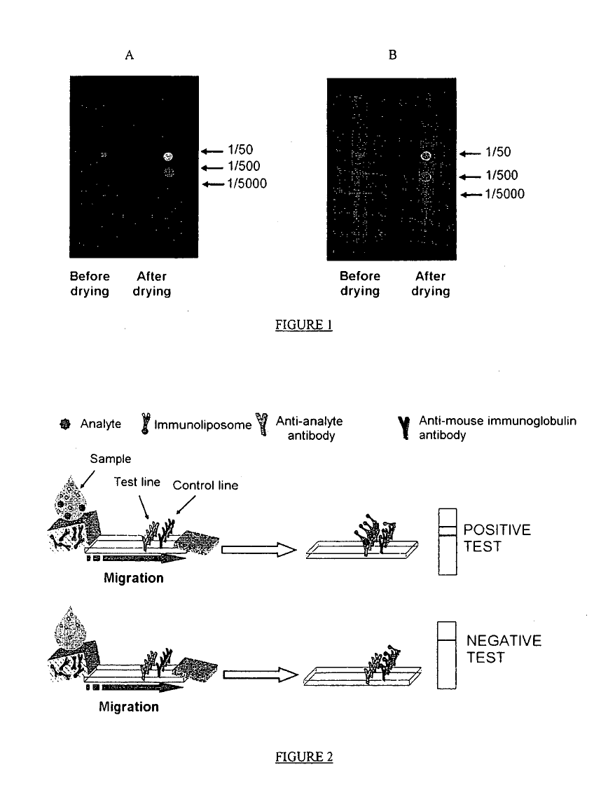

In addition to the arrangements above, the invention also comprises other arrangements which will emerge from the further description which follows, which refers to nonlimiting examples demonstrating the effect of the drying on the increase in fluorescence of the liposome-encapsulated molecules in immunoassays on a solid support, and also to the attached FIGS. 1 to 14 in which:

FIG. 1 shows the effect of the drying on the fluorescence of the liposomes;

FIG. 2 shows schematically a strip test in immunometric format;

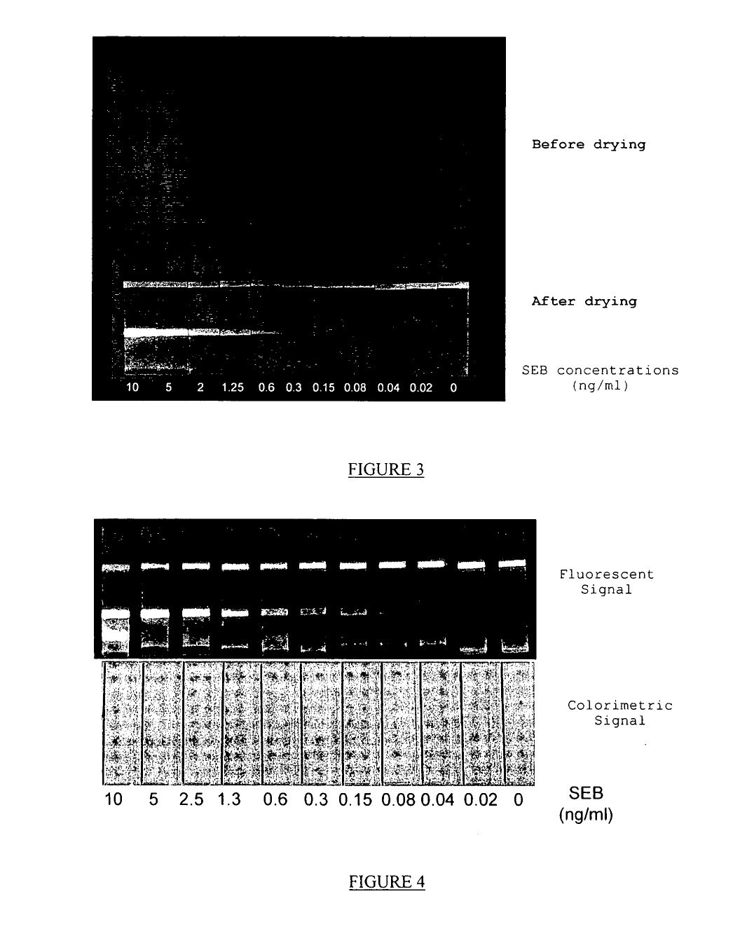

FIG. 3 shows the effect of the drying on the fluorescence of the liposomes in a strip test in immunometric format;

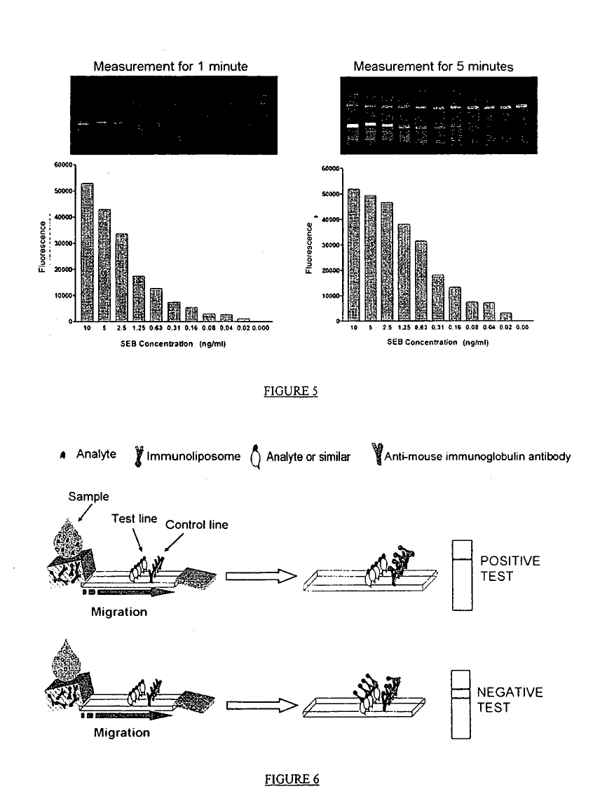

FIG. 4 shows the limits of detection obtained with liposomes with colorimetric or fluorescent measurement, in a strip test in immunometric format;

FIG. 5 shows the quantification of the fluorescent signal obtained with liposomes in a strip test in immunometric format;

FIG. 6 shows schematically a strip test in competitive format;

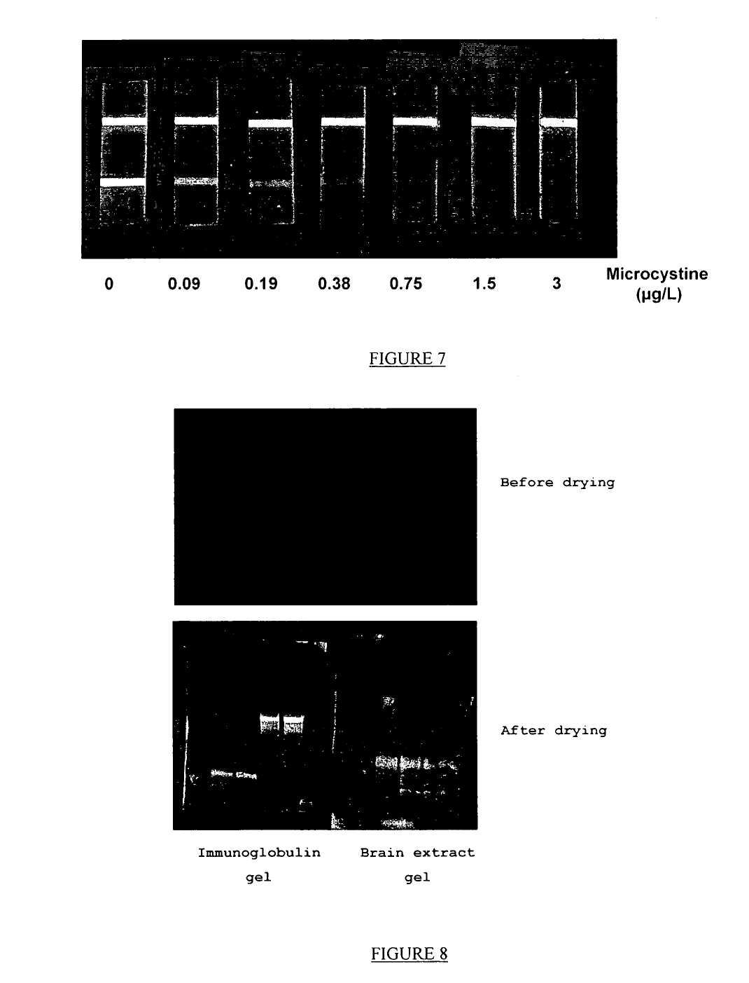

FIG. 7 shows the effect of the drying on the fluorescence of the liposomes in a strip test in competitive format;

FIG. 8 shows the effect of the drying on the fluorescence of the liposomes in an immunoblotting test;

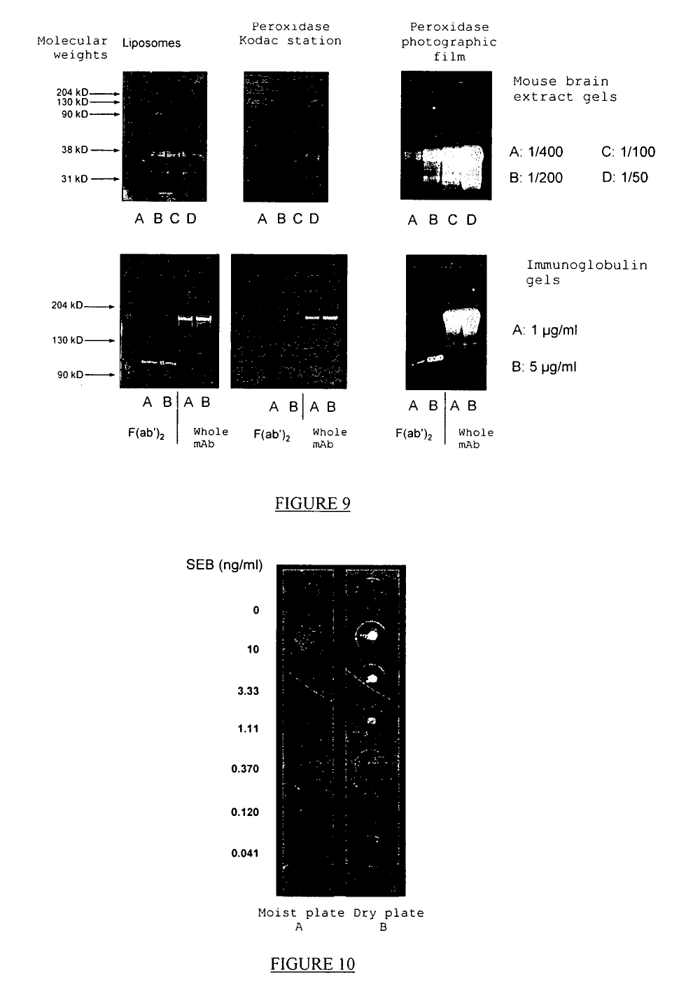

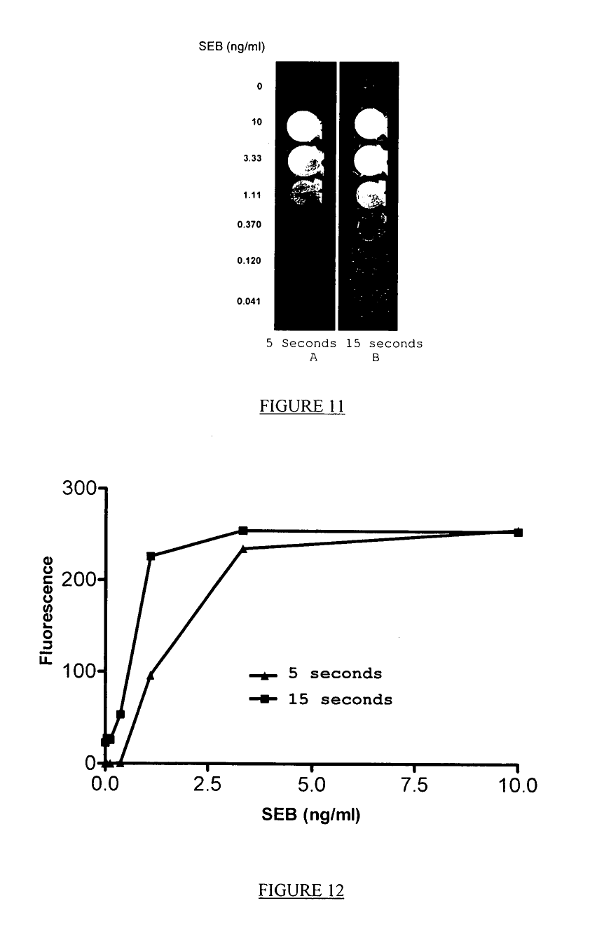

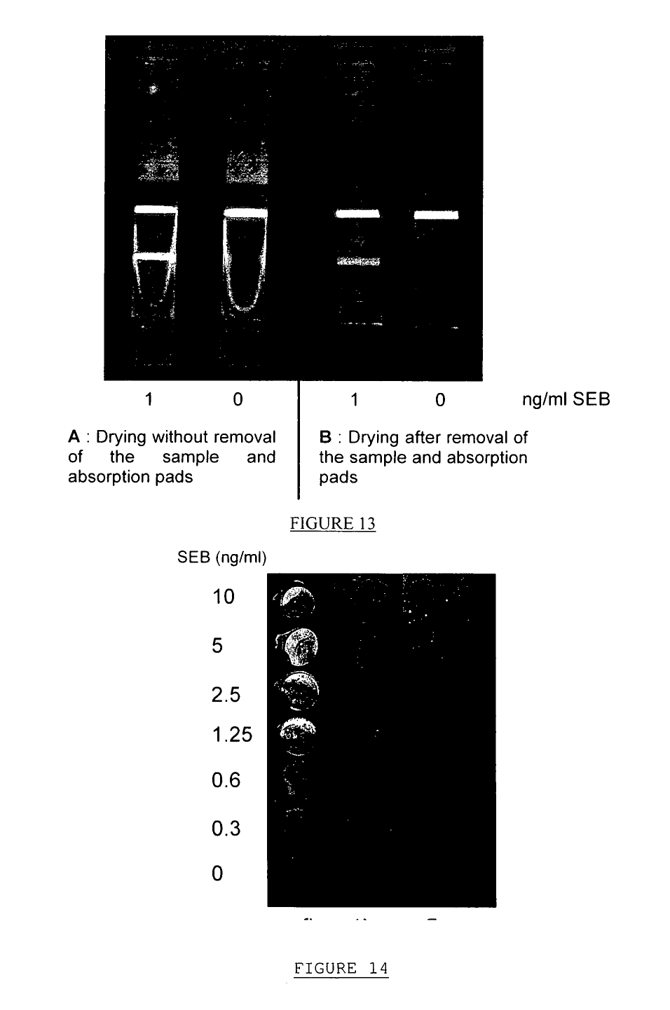

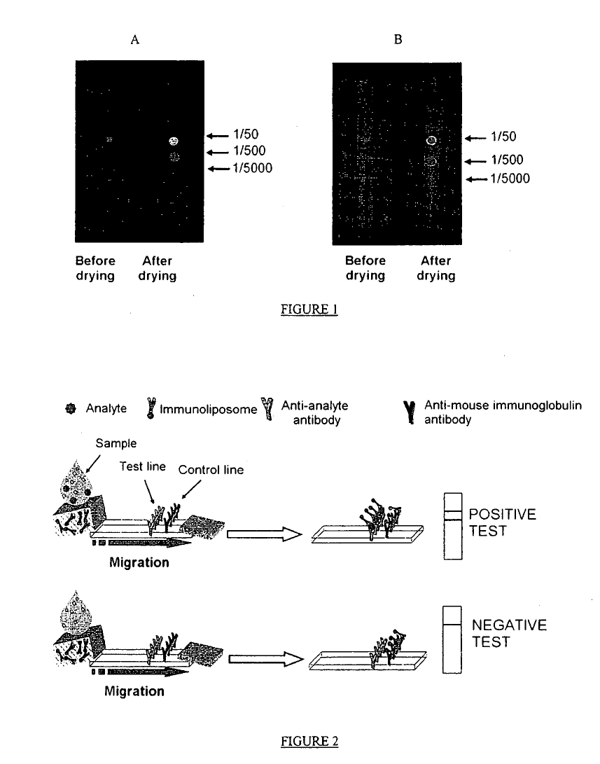

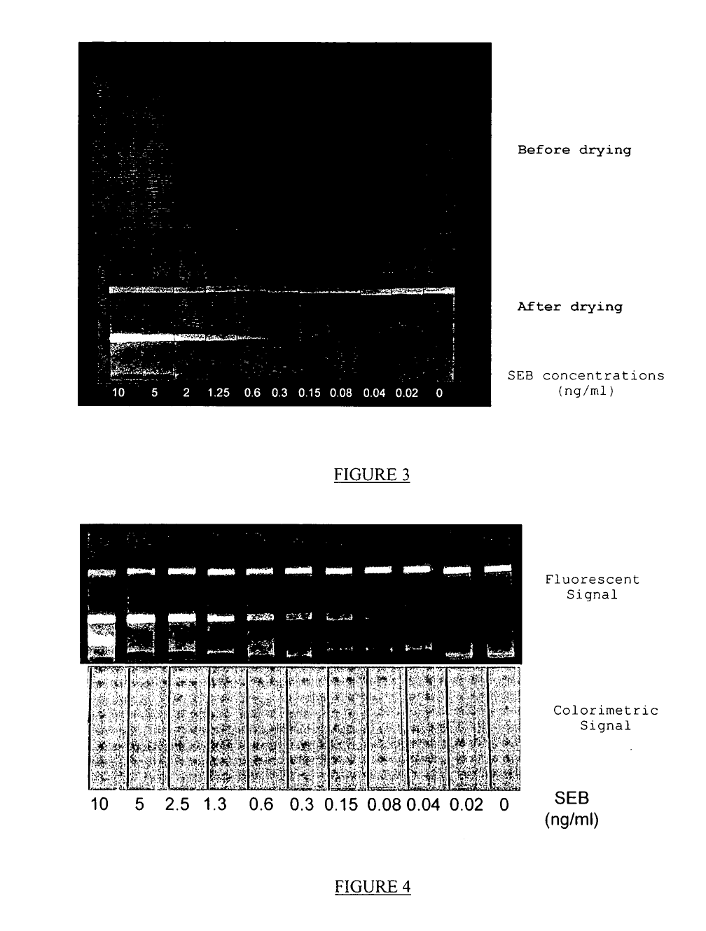

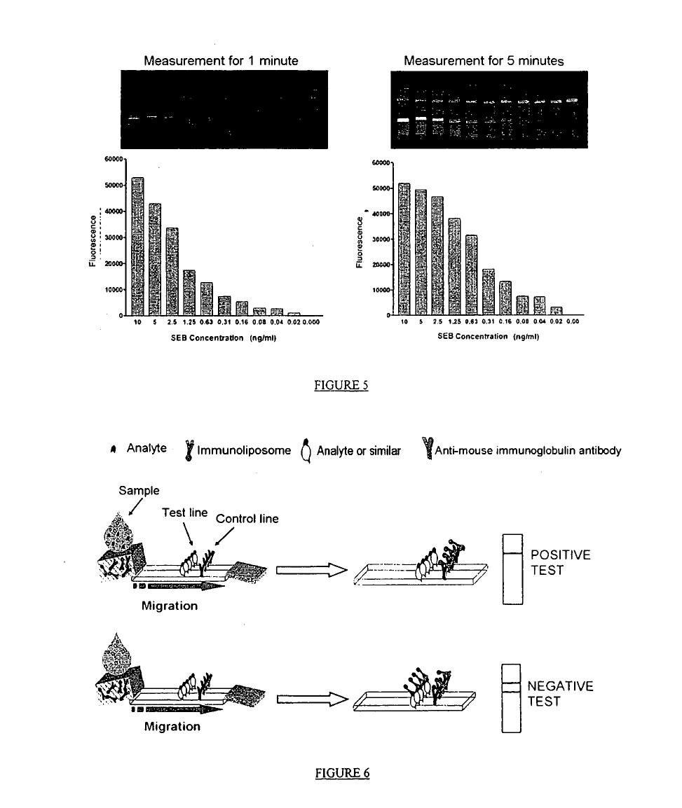

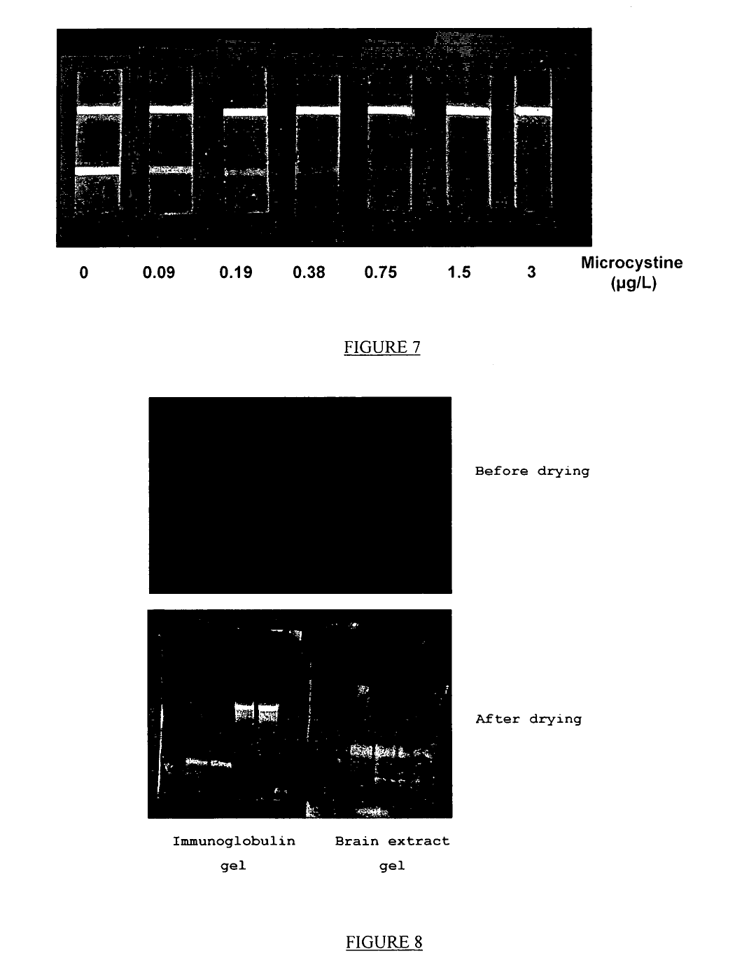

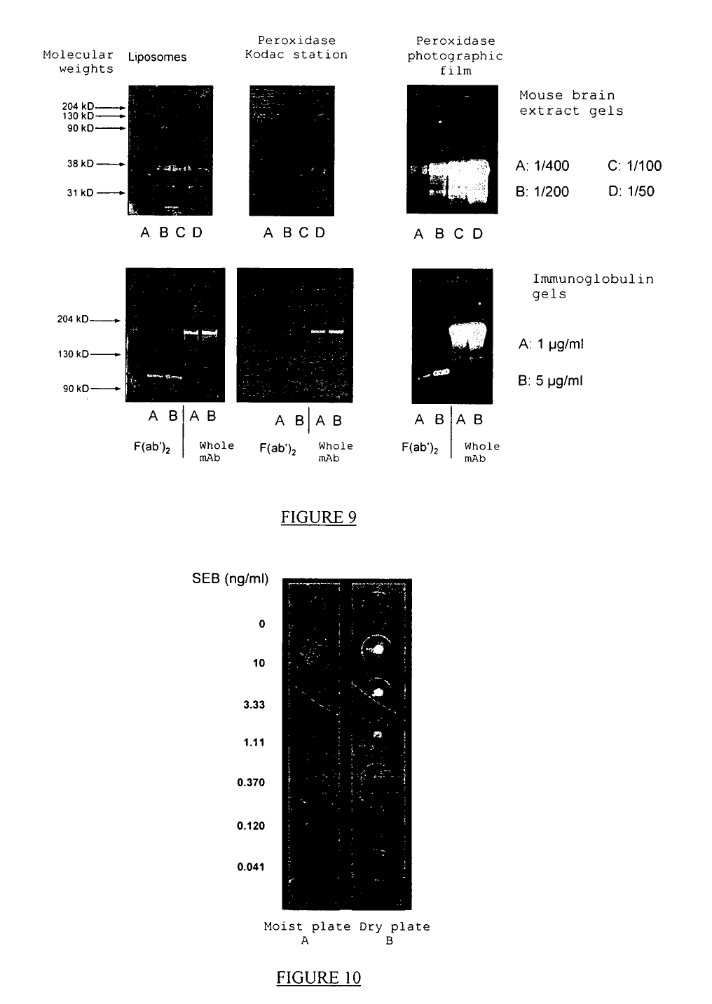

FIG. 9 shows the limits of detection obtained with liposomes with luminescent measurement (revealed or not by photographic film) or fluorescent measurement in an immunoblotting test;

FIGS. 10 and 11 show the effect of the drying on the fluorescence of the liposomes in a test in a polystyrene microtitration plate;

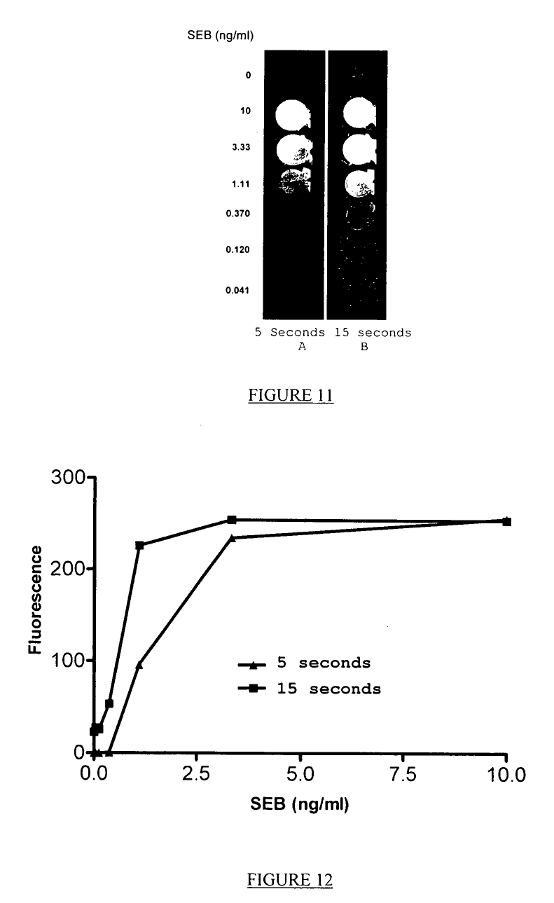

FIG. 12 shows the quantification of the fluorescent signal obtained with liposomes in a test in a polystyrene microtitration plate;

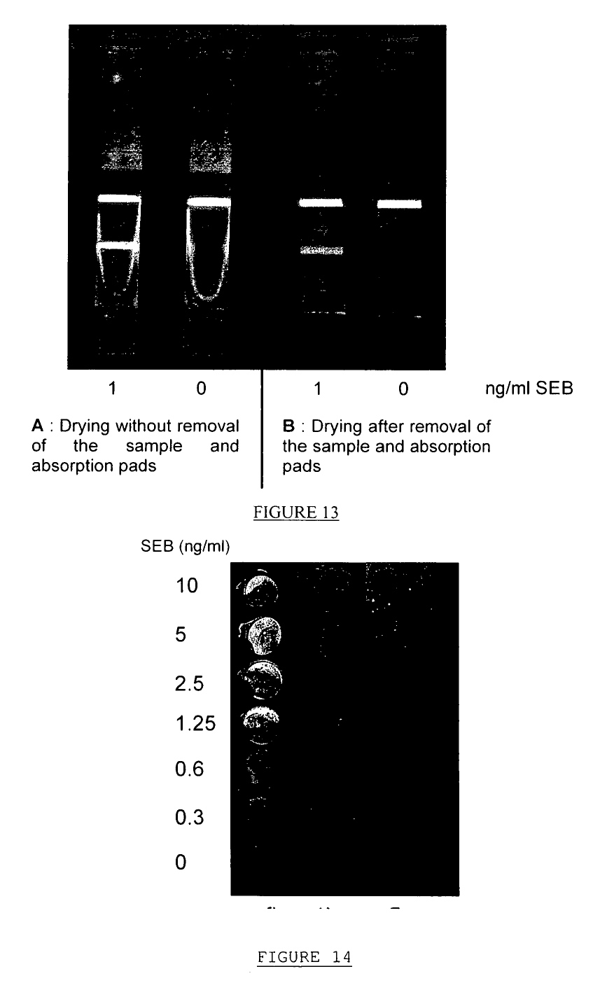

FIG. 13 is a photograph of the results of liposome fluorescence obtained in a strip test in an immunometric format. FIG. 13A: the lysis of the liposomes was carried out before completely carrying out step 2) of drying the support so as to remove the liquid medium present outside the liposomes (comparative method not part of the invention); FIG. 13B: the lysis of the liposomes was carried after having carried out step 2) of drying the support so as to remove the liquid medium present outside the liposomes (method in accordance with the invention);

FIG. 14 shows the effect of the presence or absence of a liquid medium outside the liposomes during the liposome lysis step (step 3), on the quality of the fluorescent signal in a test in a microtitration plate. In this figure, the left-hand column of wells corresponds to the wells that have been subjected to a lysis step by drying after removal of the liquid medium outside the liposomes in accordance with the method of the invention (i); the central column of wells corresponds to the wells in which the liposomes were lysed by heat shock in the presence of a liquid medium according to a comparative method not in accordance with the invention (ii); the right-hand column of wells corresponds to wells having been lysed by sonication in the presence of a liquid medium according to a comparative method not in accordance with the invention (iii).

It should be understood, however, that these examples are given purely by way of illustration of the invention, of which they in no way constitute any limitation.

### EXAMPLE 1

### Demonstration of the Effect of the Drying on the Fluorescence of the Liposomes

### a) Preparation of Liposomes Containing a Fluorophore

a-1) Preparation of Liposomes Containing a Fluorophore

Liposomes were prepared from a mixture of lipids consisting of dipalmitoyl-sn-3-glycerophosphocholine (DPPC), cholesterol (Choi.), dipalmitoyl-sn-3-glycerophosphoglycerol (DPPG) and dipalmitoyl-sn-3-glycerophosphoethanolamine (DPPE) with a molar ratio of 5:5:0.5:0.25.

A total of 100 μmol of this lipid mixture was dissolved in 10 ml of a mixture of solvents consisting of chloroform, isopropyl ether and methanol at a molar ratio of 6:6:1. After sonication of this solution at 60° C. under nitrogen, 1.5 ml of fluorophore (sulphorhodamine or Cy5®) at 150 mM were added in 0.1 M potassium phosphate buffer, pH 7.4. After sonication for 6 minutes at 60° C. under nitrogen, the organic solvents were evaporated under vacuum at 45° C. using a rotary evaporator. A gel corresponding to the suspension of liposomes was thus obtained. After having added 3 ml of fluorophore at 150 mM (sulphorhodamine or Cy5®) in 0.1 M potassium phosphate buffer, pH 7.4 to this solution, sonication was carried out for 5 minutes at 60° C. under nitrogen.

a-2) Extraction of Liposomes

In order to obtain a solution of liposomes of uniform size (0.4 μm), the liposome solution was incubated at 60° C. in a water bath and extruded through polycarbonate membranes with decreasing pore size (3 passages through filters of 1.2 μm then 3 passages through filters of 0.4 μm). The liposome solution thus obtained was centrifuged at 45,000 rpm for 30 minutes at 4° C. The pellet containing the liposomes was taken up in 0.1 M sodium phosphate buffer, pH 7, +0.15 M NaCl, and then the free (nonencapsulated) sulphorhodamine was removed by performing exclusion chromatography on Sephadex® G25 gel. At the end of this chromatography, the liposome solution was centrifuged at 45,000 rpm for 30 minutes at 4° C. The pellet thus obtained was taken up in 4 ml of 0.1 M sodium phosphate buffer, pH 7, +0.15 M NaCl, and stored at 4° C. in the dark.

b) Demonstration of the effect of the drying on the fluorescence of the liposomes

The liposomes with either sulphorhodamine or Cy5® as fluorophore were diluted in 0.1 M potassium phosphate buffer, pH 7.4, +0.15 M NaCl+0.1% BSA+0.5% Tween® 20+0.01% NaN₃, to 1/50, 1/500 and 1/5000.

5 μl of each of the dilutions were deposited on a membrane bonded to a cardboard support, and the fluorescence was then measured for 1 minute using the Image Station 4000 mM (Kodak Molecular Imaging Systems, USA) with an excitation filter at 535 nm and 625 nm for sulphorhodamine and Cy5®, respectively, and an emission filter at 600 nm and 670 nm for sulphorhodamine and Cy5®, respectively. The strips were dried for 5 minutes in an oven at 40° C. and the fluorescence was again measured.

The results are given in attached FIG. 1.

Legend of FIG. 1:
A=sulphorhodamine
B=Cy5®.

As is observed in FIG. 1, the drying greatly increases the fluorescence.

### EXAMPLE 2

### Use of the Liposomes in a Strip Test in an Immunometric Format

a) Principle of the immunometric assay

FIG. 2 shows schematically the principle of an “immunometric” assay, for which a test line consists of immobilized anti-analyte antibodies and a control line consists of immobilized anti-mouse immunoglobulin antibodies. The strips used to carry out the immunochromatographic tests consist of three components (sample pad, nitrocellulose membrane and absorption pad) bonded to a cardboard support. If the analyte sought is present in the sample, it will complex, firstly, with the liposomes added to the sample via mouse anti-analyte antibodies attached to the outer face of said liposomes, and then, secondly, during the migration on the strip, it will bind to the other anti-analyte antibodies immobilized on the test line. The correct function of the test is verified at the control line due to the binding of the mouse anti-analyte antibodies attached to the outer face of the noncomplex liposomes, to the immobilized anti-mouse immunoglobulin antibodies. The presence of the analyte being sought in the sample will be reflected by the appearance of a signal at the test line. Conversely, an absence of analyte in the sample will be reflected by an absence of signal at the test line.

### b) Preparation of the Strip Test in an Immunometric Format

b-1) Preparation of the Immunoliposomes
b-1-1) Derivation of the Liposomes with Succinimidyl Acetyl Thiopropionate (SATP)

17 μl of a solution of SATP at 10 mg/ml in dimethylformamide (DMF) were added to 300 p. 1 of a solution of liposomes with sulphorhodamine prepared as previously described in Example 1. After incubation for one hour at 20° C., the reaction was stopped by adding 100 μl of 1 M tris/HCl buffer, pH 7. After 15 minutes, the SATP that had not reacted was removed by exclusion chromatography with a Sephadex® G25 gel using 0.1 M sodium phosphate buffer, pH 7, +0.15 M NaCl. The fractions corresponding to the liposomes were mixed and centrifuged at 45,000 rpm for 30 minutes at 4° C. The pellet obtained was taken up in 225 μl of 0.1 M sodium phosphate buffer, pH 7.4, +0.15 M NaCl, and the thiol groups of the SATP were deprotected by adding 25 μl of 1 M hydroxylamine, pH 7.

b-1-2) Modification of the Mouse Antibodies with N-Succinimidyl-4-(Maleimidomethyl)Cyclohexanecarboxylate (SMCC)

A solution of SMCC at 1 mg/ml in DMF was added to a solution of murine monoclonal antibodies against Staphylococcus aureus enterotoxin B (SEB) in 0.1 M potassium phosphate buffer, pH 7.4, with a molar ratio of 20:1. After incubation for 1 hour at 20° C., the reaction was stopped by adding 1 M Tris, pH 7, for 15 minutes. The antibody thus derived was purified by exclusion chromatography on Sephadex® G25 gel with 0.1 M sodium phosphate buffer, pH 7.4, +0.15 M NaCl; the fractions containing the antibody-SMCC were determined by measuring the absorbance at 280 nm.

b-1-3) Coupling of the Liposomes with the Antibodies

The coupling of the liposomes with the antibodies was carried out by incubating 500 μg of antibody-SMCC with the deprotected liposomes-SATP (250 μl) overnight at 4° C. The noncoupled antibodies were separated from the liposomes by exclusion chromatography with Sepharose® Cl-4B gel (Sigma-Aldrich) using 0.1 M sodium phosphate buffer, pH 7.4, +0.15 M NaCl. The fractions corresponding to the liposomes were collected and centrifuged at 45,000 rpm for 30 minutes at 4° C. The pellet obtained was taken up in 500 μl of 0.1 M sodium phosphate, pH 7.4, +0.15 M NaCl+0.01% NaN₃, and stored at 4° C. in the dark.

b-2) Preparation of the Strips

The detection zone on the nitrocellulose membrane consists of two lines of reagents: a control line corresponding to the deposition of an anti-mouse immunoglobulin antibody at 500 μg/ml in 10 mM sodium phosphate buffer, pH 7.4, +0.15 M NaCl, at 1 μl/cm, using an automatic dispenser (BioDot AirJet® 3050, Irvine, Calif.), and a test line corresponding to the deposition of an anti-Staphylococcus aureus enterotoxin B (SEB) antibody at 1 mg/ml in 10 mM sodium phosphate buffer, pH 7.4, +0.15 M NaCl, at 1 μl/cm.

The membranes were dried for 1 hour at 40° C. in an oven, and were then saturated with 10 mM sodium phosphate buffer, pH 7.4, +0.15 M NaCl+0.5% of bovine albumin (BSA), for 30 minutes at ambient temperature with agitation. The membranes were then washed twice with ultrapure water for 5 minutes at ambient temperature with agitation, then incubated with 10 mM sodium phosphate buffer, pH 7.4, +0.15 M NaCl+0.15% Tween® 20, for 15 minutes at ambient temperature with agitation. The membranes were dried for 15 minutes at 40° C. in an oven, and then the sample pad was bonded to the bottom of the membrane and the absorption pad was bonded at the top of the membrane. Finally, the strips were obtained by cutting the membranes thus prepared to a width of 5 mm using a programmable guillotine (Guillotine Cutting® CM 4000, BioDot Irvine Calif.).

c) Demonstration of the effect of the drying on the fluorescence in a strip test in an immunometric format

The liposomes with the sulphorhodamine and the anti-SEB monoclonal antibodies previously obtained in paragraph b-1) were diluted to 1/500 in 0.1 M potassium phosphate buffer, pH 7.4, +0.15 M NaCl+0.01% NaN₃. A concentration range for SEB (Sigma-Aldrich) (10; 5; 2.5; 1.25; 0.6; 0.3; 0.15; 0.08; 0.04; 0.02 and 0 ng/ml) was prepared in 0.1 M potassium phosphate buffer, pH 7.4, +0.15 M NaCl+0.1% BSA+0.5% Tween° 20+0.01% NaN₃.

100 μl of each of the concentrations of SEB with 10 μl of liposomes were deposited into microtitration plate wells. After incubation for 10 minutes, with agitation at ambient temperature and in the dark, a strip prepared as described in paragraph b-1) (with a second anti-SEB monoclonal antibody immobilized at the test line) was placed in the wells vertically (sample pad in contact with the solution) and the migration took place by capillary action. After the solution had completely migrated on the strips, these ones were removed from the wells.

The fluorescence was measured for one minute using the Image Station® 4000 mM (Kodak Molecular Imaging Systems, USA) with an excitation filter at 535 nm and an emission filter at 600 nm. The strips, after having removed the sample pads and the absorption pads, underwent drying for 5 minutes at 40° C. in an oven (steps 2 and 3) and the fluorescence was measured under the same conditions as previously.

The results are given in FIG. 3.

The results show that the drying makes it possible to obtain a fluorescent signal at the test and control lines. Furthermore, the signal at the test line increases with the concentration of SEB, which is in agreement with the principle of the immunometric format.

This experiment demonstrates that removing the liquid and then drying are essential for using liposomes as fluorescent labels in strip tests.

d) Comparison of the detection limits obtained with liposomes with colorimetric or fluorescent measurement in an immunometric format

Liposomes with sulphorhodamine as fluorophore and anti-SEB monoclonal antibodies were prepared as previously described in paragraph b-1), and then diluted to 1/50 in 0.1 M potassium phosphate buffer, pH 7.4, +0.15 M NaCl+0.01% NaN₃. A concentration range for SEB (Sigma-Aldrich) was also prepared as described above in paragraph c).

The bringing of the liposomes into contact with the various concentrations of SEB and then the migration by capillary action were carried out as previously described in paragraph c).

After the solution had completely migrated on the strips, the sample pads and the absorption pads were removed, the strips were dried for 5 minutes at 40° C. (steps 2 and 3) and the fluorescence was measured as previously described in paragraph c), and then these same strips were scanned with an Epson Expression® 1640XL scanner in order to measure the colorimetric signal.

The results are given in FIG. 4.

The results show that the detection limit obtained with the fluorescent signal is approximately 20 pg/ml, whereas it is approximately 300 pg/ml with the colorimetric signal.

The use of the fluorescent signal by means of the drying method therefore makes it possible to increase the detection limit by a factor of 15 using the same reagents under the same conditions.

e) Example of quantification of the fluorescent signal in a strip test in an immunometric format

Liposomes with sulphorhodamine as fluorophore and anti-SEB monoclonal antibodies were prepared, and then diluted to 1/500 as previously described in paragraph c). A concentration range for SEB (Sigma-Aldrich) was also prepared as previously described in paragraph c).

The bringing of the liposomes into contact with the various concentrations of SEB and then the migration by capillary action were carried out as previously described in paragraph c).

After the solution had completely migrated on the strips, the fluorescence was measured for one minute as previously described in paragraph c). The sample pads and the absorption pads were removed, the strips were then dried for 5 minutes at 40° C. (steps 2 and 3) and the fluorescence was measured for 1 minute and 5 minutes as previously described in paragraph c). In order to quantify the fluorescent signal corresponding to each concentration, the fluorescent signal of a zone of the same dimensions located between the test line and the control line of the same strip was subtracted from the fluorescent signal of the test line. All these quantifications were carried out using the Image J software (Rasband, W. S., Image J. U.S. National Institutes of Health, Bethesda, Md. USA, http://rsb.info.nih.gov/ij/, 1997-2006).

The results are given in FIG. 5.

The results show that measuring the fluorescence for a longer period of time makes it possible to increase the fluorescent signals, thereby increasing the detection limit. Thus, the reading time can be modulated according to the results obtained and the desired detection limits.

### EXAMPLE 3

### Use of the Liposomes in a Strip Test in Competitive Format

a) Principle of the competitive format

FIG. 6 shows schematically the principle of a “competitive” assay for which a test line consists of an immobilized analyte or an immobilized analogue thereof, and a control line consists of immobilized anti-mouse immunoglobulin antibodies. If the analyte sought is present in the sample, it will firstly bind to the mouse anti-analyte antibodies attached to the outer face of the immunoliposomes that will therefore no longer be able to bind to the analyte or to its analogue immobilized at the test line during the migration on the strip since the binding sites will be occupied. The presence of the analyte being sought in the sample will therefore be reflected by a decrease in or a total disappearance of the signal at the test line. Conversely, if the analyte being sought in the sample is absent, this will allow the liposomes to bind to the analyte or to its analogue immobilized at the test line, and will therefore be reflected by the presence of a signal at the test line. In all cases, the signal at the control line remains at a maximum and unchanged due to the binding of the liposomes to the immobilized anti-mouse immunoglobulin antibodies via the mouse anti-analyte antibodies attached to the outer face of the immunoliposomes.

b) Preparation of the strip test
b-1) Preparation of the Immunoliposomes

The liposomes are prepared as previously described in paragraph b-1) of Example 2, except that a solution of anti-microcystine LR murine monoclonal antibodies in 0.1 M potassium phosphate buffer, pH 7.4, was used.

b-2) Preparation of The Strips

The strips used are prepared as previously described in paragraph b-2) of Example 2, except that the test line corresponds to the deposition of microcystine LR coupled to bovine albumin at 5 μg/ml in 10 mM sodium phosphate buffer, pH 7.4, +0.15 M NaCl, at 1 μl/cm.

### c) Strip Test in Competitive Format

The liposomes with the sulphorhodamine as fluorophore and anti-microcystine LR monoclonal antibodies thus obtained were diluted to 1/500 as previously described in paragraph c) of Example 2. A concentration range for microcystine LR (Sigma-Aldrich) (3; 1.5; 0.75; 0.38; 0.19; 0.09 and 0 ng/ml) was prepared in 0.1 M potassium phosphate buffer, pH 7.4, +0.15 M NaCl+0.1% BSA+0.5% Tween20+0.01% NaN₃.

100 μl of each of the concentrations of microcystine LR with 10 μl of liposomes were deposited into microtitration plate wells. After incubation for 10 minutes, a strip (with microcystine coupled to BSA immobilized at the test line) was placed in the wells vertically. After the solution had completely migrated on the strips, the sample pads and the absorption pads were removed, the strips were dried for 5 minutes at 40° C. (steps 2 and 3) and the fluorescence was measured for one minute under the same conditions as those previously described in paragraph c) of Example 2.

The results are given in FIG. 7.

The results obtained show that the liposomes can be used irrespective of the detection format used. The fluorescent signals are inversely proportional to the concentration of microcystine, which is in agreement with the competitive format.

### EXAMPLE 4

### Other Application of the Liposomes on a Membrane: Western Blotting

Samples either of mouse brain extract for detecting prion protein, or of whole purified mouse immunoglobulins or purified mouse immunoglobulins in the form of F(ab′)₂ fragments were used. The mouse brain extract was diluted to 1/50, 1/100, 1/200 and 1/400 in Laemmli buffer (60 mM Tris/HCl, pH 6.8, +0.1% SDS+10% glycerol+0.3% bromophenol blue) containing 2.5% of βmercaptoethanol. The immunoglobulins were diluted to 1 μg/ml and 5 μg/ml in Laemmli buffer.

The brain extracts were loaded (15 μl/dilution) onto a 12% polyacrylamide gel, and the immunoglobulins (15 μl/dilution) onto a 6% polyacrylamide gel. They were loaded in duplicate. The electrophoresis was carried out for 1 hour at 200 volts with a Tris/glycine migration buffer.

The electrotransfer of the molecules present in the polyacrylamide gels was carried out on a PolyVinylidene DiFluoride (PVDF) membrane sold under the trade name Immobilon-FL® (Millipore) in Tris/CAPS buffer (Biorad) at 100 volts for 1 hour.

The membrane was recovered and rinsed by rapid immersion in 0.1 M sodium phosphate buffer, pH 7.4, +0.15 M NaCl (PBS), and then in ethanol, and finally in water for 5 minutes. The membrane was saturated for 30 minutes in PBS+0.1% Tween® 20+5% BSA. The membrane was rinsed by two short washes (10 seconds) and then one of 5 minutes with PBS+0.1% Tween 20.

The membrane corresponding to the mouse brain extracts was preincubated for 30 minutes at ambient temperature with an anti-prion protein antibody diluted to 4 μg/ml in PBS+0.1% Tween® 20+1% BSA, and then rinsed by rapid immersion followed by two washes for 5 and 10 minutes in PBS+0.1% Tween 20.

The membranes corresponding to the brain extracts and to the immunoglobulins were then incubated either with the anti-mouse immunoglobulin antibody coupled to peroxidase (Pierce) diluted to 1/1000 in PBS+0.1% Tween20+3% BSA, or with the liposomes containing sulphorhodamine and coupled to an anti-mouse immunoglobulin antibody, diluted to 1/500 in PBS+0.1% Tween® 20+3% BSA, for 20 minutes at ambient temperature. The membranes were subsequently rinsed by rapid immersion followed by one wash for 5 minutes and two washes for 10 minutes in PBS+0.1% Tween® 20. Finally, the membranes were rinsed in PBS for 1 minute.

Subsequently, the membranes corresponding to the liposomes were dried for 5 minutes at 40° C. (steps 2 and 3) and the fluorescence was measured for 1 minute (FIG. 8) and 5 minutes (FIG. 9).

The membranes corresponding to the peroxidase were immersed in ECL Plus® (Pierce) and then placed under a plastic film, and the luminescence was read for one minute (FIG. 8) and 5 minutes (FIG. 9) without an excitation or emission filter, using the Kodak® 2000 mM image station. After this measurement, the luminescence was also revealed using a photographic film according to a conventional procedure (FIG. 9).

The results are given in FIGS. 8 and 9.

As shown in FIG. 8 (signal scale from 0 to 2500), drying of the membranes prior to liposome lysis is essential for obtaining a localized fluorescent signal of good quality, thereby confirming the results obtained with the strips.

As shown in FIG. 9 “liposomes” (signal scale from 0 to 16,000), the liposomes make it possible to obtain a sensitivity that is at least equivalent to that of luminescence revealed by photographic film (FIG. 9 “peroxidase photographic film”, scan of the photographic film processed by Image J) and much higher than that obtained with luminescence measured using the Kodak® station (FIG. 9 “peroxidase Kodak® station”, signal scale from 0 to 160). In addition, the bands corresponding to the various molecules revealed by the antibodies are much less diffuse with the immunoliposomes, which facilitates the analysis thereof and makes said analysis more accurate.

### EXAMPLE 5

### Use of the Liposomes in Microtitration Plates

Polystyrene microtitration plates (Colstar) in which the wells have black walls but a transparent bottom were used. The polystyrene of these plates has a high protein-adsorption capacity.

1 μl of a solution of neutravidin (avidin analogue sold by Pierce) at 1 mg/ml in 0.05 M phosphate buffer, pH 7.4, was deposited on the bottom of the wells. After incubation for 16 hours at 20° C. in a humid chamber, the neutravidin solution was removed and the plates were washed with 0.1 M phosphate buffer, pH 7.4, +0.1% Tween 20 (washing buffer).

The adsorption sites on the plates were saturated by depositing in the wells 300 μl of a solution of 0.1 M phosphate buffer, pH 7.4, +0.1% BSA+0.15 M NaCl+0.01% NaN₃ (dilution buffer). After washing the plates with phosphate buffer, 40 μl at 1 μg/ml of a biotinylated first anti-enterotoxin B (SEB) antibody were deposited in the wells, for 30 minutes at 20° C.

In parallel, a range of SEB (10; 3.33; 1.11; 0.37; 0.12; 0.041 and 0 ng/ml) in dilution buffer was mixed (v/v) with liposomes (containing sulphorhodamine B) diluted to 1/500 in 0.1 M phosphate buffer, pH 7.4, +0.15 M NaCl, to which a second anti-SEB antibody was coupled. After incubation for 30 minutes at 20° C., and after washing of the plates with washing buffer, 40 μl of these solutions were deposited in the wells for 30 minutes at 20° C. The plates were subsequently washed, then 100 p. 1 of 0.1 M phosphate buffer, pH 7.4, +0.15 M NaCl were deposited, and the fluorescence was measured on the Kodak® Station (excitation filter at 535 nm and emission filter at 600 nm) for 1 minute (FIG. 10A). After this reading, the wells were emptied and the plate was dried for 10 minutes at 40° C. in an oven. A further reading was carried out on the Kodak® Station for 1 minute (FIG. 10B), and the fluorescence of each well was measured for 5 sec. (FIG. 11A) and 15 sec. (FIG. 11B), using an inverted microscope (Olympus® IX 71) coupled to a PCO 1600 camera (Photon Lines), thereby making it possible to plot the curves (FIG. 12) expressing the amount of fluorescence measured (in arbitrary units) as a function of the concentration of SEB (in ng/ml) at 5 seconds (solid triangles) and at 15 seconds (solid squares).

The results of FIG. 10 show that, as for the other solid supports, the drying is necessary in order to observe the appearance of a fluorescent signal.

Furthermore, the results of FIG. 11 show that the fluorescent signal is completely located at the spots. Finally, the results of FIG. 12 show that the signal obtained is quantifiable and proportional to the amount of analyte present in the sample. All these observations make it possible to envisage the use of the liposomes with biochips consisting of several thousand spots, and analysing either polynucleotides, or peptides or proteins.

Thus, a subject of the invention is also the use of the detection and quantification method as defined above, for reading biochips.

### EXAMPLE 6

### Demonstration of the Importance of the Procedure Used for Drying the Supports and Lysing the Liposomes

### a) Use of the Liposomes in a Strip Test

Liposomes with sulphorhodamine and anti-SEB monoclonal antibodies were prepared as previously described in paragraph b-1) of Example 2. These liposomes were subsequently diluted to 1/500 in 0.1 M potassium phosphate buffer, pH 7.4, +0.015 M NaCl+0.01% NaN₃. Two concentrations (5 and 0 ng/ml) of enterotoxin B (Sigma Aldrich) were prepared in 0.1 M potassium phosphate buffer, pH 7.4, +0.15 M NaCl+0.1% BSA+0.5% Tween® 20+0.01% NaN₃. 100 μl of each concentration of SEB were deposited in microtitration plate wells with 10 μl of liposomes. After incubation for 10 minutes, a strip (with a second anti-SEB monoclonal antibody immobilized at the test line) was placed in the wells vertically. Two strips were prepared for each concentration of enterotoxin B. After the solution had completely migrated on the strips, the sample pads and the absorption pads were removed from one of the strips of each of the concentrations, and the strips were dried at 40° C. for 45 minutes.

For the other strip of each of the concentrations, the pads were not removed and the strips were left to dry in an oven at 40° C. for 45 minutes. The drying time for the strips was longer than previously since it takes much longer to dry the pads than to dry the membrane alone (approximately 5 minutes).

The fluorescence was then read under the same conditions as previously indicated above in Example 2) c).

The results are given in attached FIG. 13, in which the results obtained for the two strips on the left (FIG. 13 A) are those obtained with the strips for which the pads were not removed before the drying step, whereas the results obtained for the two strips on the right (FIG. 13 B) are those obtained with the strips for which the pads were removed before the drying step.

These results show that, when the liposome lysis takes place even though the liquid medium present outside the liposomes has not been completely removed, there is an uncontrolled diffusion of the signal on the strip. This phenomenon is due to nonhomogeneous drying. This is because the strip dries more rapidly than the pads, thereby inducing lysis of the liposomes and migration of liquid originating from the pads, which will cause an uncontrolled diffusion of the free fluorophores on the strip.

### b) Use of the Liposomes in Microtitration Plates

To carry out this study, polystyrene microtitration plates (Costar) in which the wells have black walls but a transparent bottom were used. The polystyrene of these plates has a high protein-adsorption capacity. 1 μl of a solution of NeutrAvidin® (avidin analogue sold by the company Pierce, USA) at a concentration of 1 mg/ml in 0.05 M phosphate buffer, pH 7.4, was deposited on the bottom of the wells. After incubation for 16 hours at 20° C. in a humid chamber, the NeutrAvidin® solution was removed and the plates were washed with 0.1 M phosphate buffer, pH 7.4, +0.01% Tween® 20 (washing buffer).

The adsorption sites on the plates were saturated by depositing into the wells 300 μl of a solution of 0.1 M phosphate buffer, pH 7.4, +0.1% bovine albumin+0.15 M NaCl+0.01% NaN₃ (dilution buffer). After washing of the plates with washing buffer, 40 μl of a solution at 1 μg/ml of a biotinylated first anti-enterotoxin B antibody were deposited in the wells for 30 minutes at 20° C. In parallel, a range of enterotoxin B (10; 5; 2.5; 1.25; 0.6; 0.3 and 0 ng/ml) in dilution buffer was mixed (v/v) with liposomes containing sulphorhodamine B (as prepared in step b-1) of Example 2 above) and diluted to 1/500 in 0.1 M phosphate buffer, pH 7.4, +0.15 M NaCl, to which a second anti-enterotoxin B (SEB) antibody was coupled as described above in Example 2, steps b-1-2) and b-1-3). After incubation for 30 minutes at 20° C. and after washing of the plates with washing buffer, 40 μl of these solutions were deposited in the wells for 30 minutes at 20° C. Each concentration was deposited in three separate wells. The plates were subsequently washed and the three wells corresponding to the same concentration were treated differently:

i) one well was left empty (for liposome lysis by subsequent drying in the absence of liquid medium outside the immunoliposomes, in accordance with the method of the invention),

ii) 40 μl of 0.1 M phosphate buffer, pH 7.4, +0.15 M NaCl were added, at 100° C., to the second well (in order to carry out the liposome lysis by heat shock and then to dry them in the presence of buffer: comparative method not part of the invention),

iii) and the third well was subjected to ultrasound for 10 seconds after having deposited therein 40 μl of 0.1 M phosphate buffer, pH 7.4, +0.15 M NaCl (to carry out the liposome lysis by the action of ultrasound before the drying step in the presence of buffer: comparative method not part of the invention).

All the wells were then dried for 3 hours at 40° C. in an oven. A longer drying time than previously (Example 5) is necessary in order to completely dry the wells containing 40 μl of buffer. The fluorescence of each well was measured for 5 seconds using an inverted microscope (Olympus IX 71) coupled to a pco. 1600 camera (sold by the company Photon Lines).

The results are represented in attached FIG. 14.

In this figure:
the column of wells on the left corresponds to the wells having undergone a drying step after removal of the liquid medium present outside the immunoliposomes, in accordance with the method of the invention (i);
the central column of wells corresponds to the wells having been dried after liposome lysis by heat shock in liquid medium (ii);
the column of wells on the right corresponds to the wells having been dried after liposome lysis by sonication in liquid medium (iii).

The results obtained show that the liposome lysis is not by itself sufficient to obtain a localized fluorescent signal. In fact, it is observed on FIG. 14 that the liposome lysis by heat shock or ultrasound in liquid medium induces a large decrease or even complete disappearance of the fluorescent signal, whereas drying the liposomes in the absence of liquid medium makes it possible to obtain a very well-localized fluorescent signal.

In conclusion, the step of revealing the fluorescent signal should be carried out according to a protocol and treatment suitable for the solid support used, in order for the liposome lysis, irrespective of the treatment used for this, to be carried out in a liquid-free environment, i.e. an environment which does not allow diffusion of the fluorophores.

1. A method for detecting and quantifying at least one analyte A in a biological sample, on a solid support, with liposomes encapsulating fluorescent molecules, said method comprising:
1) either (a) i) bringing said biological sample capable of comprising at least one analyte A into contact, in the liquid medium, with at least one liposome encapsulating a fluorescent molecule and the surface of which is coupled to molecules capable of complexing said analyte A, then ii) bringing said medium into contact on a solid support containing at least one immobilized substrate capable of binding the analyte A/liposome complexes possibly formed,
or b) bringing said biological sample pre-immobilized on a solid support, said sample being capable of comprising at least one analyte A, into contact with a liquid medium comprising said liposome encapsulating a fluorescent molecule and the surface of which is coupled to molecules capable of complexing said analyte A;
2) removing the liquid medium present outside the liposomes by drying the said solid support;
3) lysing the liposomes; and
4) detecting the fluorescent signals possibly emitted.

2. The method according to claim 1, wherein said step 1)(a)(ii) is carried out:
either by migration of said medium on said solid support due to capillary action;
or by deposition of said medium on said solid support.

3. The method according to claim 1, wherein said step 1)(b) comprises the following substeps:
i) separating at least one analyte A from said biological sample by electrophoresis, and immobilizing said analyte A on a solid support by electrotransfer; and
ii) bringing said solid support on which said analyte A is immobilized into contact with a liquid medium comprising at least one liposome encapsulating a fluorescent molecule and the surface of which is coupled to molecules capable of complexing said analyte A.

4. The method according to claim 1, wherein it also comprises, between steps 1) and 2), at least one step of washing the solid support in order to remove the noncomplexed liposomes.

5. The method according to claim 1, wherein the support is chosen from nitrocellulose membranes, microtitration plates and biochips.

6. The method according to claim 1, wherein the molecules coupled to the liposomes and capable of complexing an analyte A are chosen from primary and secondary antibodies capable of complexing said analyte, a receptor for said analyte, polynucleic acids, peptide nucleic acids, lectin, and a carrier protein or a chelate or synthetic receptor for said analyte.

7. The method according to claim 1, wherein the fluorescent molecules are chosen from phycoerythrin, phycocyanin, fluorescein and its derivatives, rhodamine and its derivatives, water-soluble derivatives of rhodamine which are in the form of an N hydroxysuccinimide ester, coumarins and their derivatives, fluorescent cyanins, sulphorhodamine derivatives and green fluorescent protein.

8. The method according to claim 7, wherein the fluorescent molecules are chosen from sulphorhodamine and fluorescent cyanins.

9. The method according to claim 1, wherein the drying step 2) is carried out at a temperature of from 20 to 90° C.

10. The method according to claim 1, wherein the duration of the drying step 2) ranges from 1 to 60 minutes.

11. The method according to claim 1, wherein the lysis of the liposomes in step 3) is carried out by additional drying of the support.

12. The method according to claim 11, wherein the duration of the additional drying ranges from 1 to 15 minutes.

13. The method according to claim 11, wherein the drying of the support in step 3) is carried out in an oven, at a temperature of from 20 to 90° C.

14. The method according to claim 1, wherein the detection of the fluorescent signals of step 4) is carried out for a period of at least 1 minute.

15. A method of reading biochips comprising employing the method according to claim 1.
